(19) United States
(12) Patent Application Publication
(10) Pub. No.: US 2026/0334777 A1
(43) Pub. Date: Oct. 29, 2026

(54) METHOD AND SYSTEM FOR DETERMINING TEAR-BREAKUP TIME

(71) Applicant: Forus Health Pvt. Ltd., Karnataka (IN)

(72) Inventors: Venkatakrishnan SRINIVASAN, Karnataka (IN); Spoorthi KULLAMBETTU, Karnataka (IN)

(73) Assignee: Forus Health Pvt. Ltd., Karnataka (IN)

(21) Appl. No.: 19/552,791

(22) Filed: Feb. 27, 2026

(30) Foreign Application Priority Data
Apr. 26, 2025 (IN) 202541040454

(51) Int. Cl. A61B 3/10; A61B 3/14; G16H 30/40

(52) U.S. Cl. CPC A61B 3/101; A61B 3/145; G16H 30/40

(57) ABSTRACT

A method and system for determining tear-breakup time. Method comprising projecting concentric rings pattern, using concentric rings generator, on exterior surface of subject’s eyeball. Method comprising capturing a video of subject’s eyeball from substantially in front of the subject’s eyeball. Method comprising identifying, using an Artificial Intelligence (AI) model, a first appearance of breakup pattern in the concentric rings pattern caused by breakup of tear film on external surface of subject’s eyeball. Method comprising tear-breakup time based on a timestamp associated with frame of video at first appearance of breakup pattern and a timestamp associated with previous blink of subject’s eyelid, from captured video.

### CROSS-REFERENCE TO RELATED APPLICATIONS

This application claims the benefit of priority from Indian Patent Application No. 202541040454, Apr. 26, 2025, the content of which is hereby incorporated by reference.

### FIELD OF THE INVENTION

The present disclosure relates to tear film analysis, and more particularly relates to a method and a system for determining tear-breakup time.

### BACKGROUND OF THE INVENTION

Dry eye disease (DED) is increasingly recognized as a prevalent issue affecting millions of people globally. The DED occurs when there is insufficient lubrication on the surface of the eye, often leading to discomfort, irritation, and potential damage to the ocular surface. A tear film, which is composed of a lipid, aqueous, and mucin layer, serves as a protective barrier to the cornea, ensuring comfort, clear vision, and protection from environmental factors. The tear breakup refers to the moment when the tear film becomes unstable, leading to the formation of dry spots on the corneal surface.

Computing Tear Breakup Time (TBUT) is one of the most critical methods for assessing dry eye conditions. The TBUT is a measure of the stability of the tear film, which plays a vital role in diagnosing the severity and underlying causes of dry eye disease. The TBUT is the interval from the initiation of a blink to the appearance of the first dry spot on the ocular surface, and it is used to assess the quality and stability of the tear film.

Despite its importance, there are several challenges associated with accurately identifying TBUT. These challenges stem from both physiological variability and limitations of available diagnostic methods. Several methods are used to calculate TBUT, for example, invasive methods and non-invasive methods, each varying in accuracy, and technology.

The traditional invasive method involves the use of fluorescein dye, where a drop of dye is applied to the ocular surface, and the time is measured from the last blink until the appearance of the first dry spot on the tear film under a slit-lamp with a cobalt blue filter. While widely used, the application of dye can cause discomfort, leading to excessive blinking or tearing that can affect the accuracy of the measurement. Therefore, to address such problems, non-invasive methods have been developed. One common non-invasive technique is tear film interferometry, where devices measure the thickness of the lipid layer of the tear film, detecting when it begins to destabilize. Optical coherence tomography (OCT) is another non-invasive method, providing high-resolution images of the tear film and corneal surface, allowing the detection of TBUT by observing when the tear film starts to break up. High-definition video systems, such as the Keratograph, capture real-time footage of the tear film’s behavior, and the software automatically analyzes the breakup time based on changes in the tear film. While traditional methods like fluorescein dye remain popular, non-invasive techniques are gaining traction due to their comfort and accuracy, offering a more objective and reliable assessment of TBUT.

However, the existing TBUT identification methods for determining the tear film breakup pattern may consist of various artifacts which can easily lead to misinterpretation of the tear film’s true breakup. For example, blinking can cause transient distortions in the tear film that may appear as if the tear film is breaking up, when in fact, the disturbance is simply due to the mechanical action of the eyelid. In such cases, the natural tear film disruption caused by blinking may be mistaken for genuine tear film instability, leading to inaccurate TBUT readings. Illumination inconsistencies may be another common issue. The lighting used during existing TBUT testing methods, especially with slit-lamp examination, can vary in intensity or angle, potentially creating shadows or uneven illumination on the corneal surface. These inconsistencies can result in false visual cues, making the existing TBUT testing methods difficult to distinguish between actual tear film breakup and superficial optical effects. Additionally, reflections from eyelashes or other ocular structures can also interfere with the clarity of the tear film’s appearance, causing reflections that may be interpreted as tear breakup, further complicating the interpretation of results. Such factors highlight the need for precise control over testing conditions and advanced technologies that can filter out such artifacts to provide more reliable and accurate TBUT measurements.

Hence, there is a need for a technique to overcome the above-discussed problems.

### SUMMARY

This summary is provided to introduce a selection of concepts, in a simplified format, that are further described in the detailed description of the invention. This summary is neither intended to identify key or essential inventive concepts of the invention nor is it intended for determining the scope of the invention.

According to one embodiment of the present disclosure, a method for determining tear-breakup time. The method includes projecting a concentric rings pattern, of light and darkness, on an external surface of a subject’s eyeball, using a concentric rings generator of light and darkness. The method includes capturing a video of the subject’s eyeball from substantially in front of the subject’s eyeball. The method includes identifying, using an Artificial Intelligence (AI) model, a first appearance of a breakup pattern in the concentric rings pattern caused by a breakup of a tear film on the external surface of the subject’s eyeball. The method includes determining the tear-breakup time based on a timestamp associated with a frame of the video at the first appearance of the breakup pattern and a timestamp associated with a previous blink of a subject’s eyelid, from the captured video.

According to one embodiment of the present disclosure, a system for determining tear-breakup time. The system includes a concentric rings generator of light and darkness configured to project a concentric rings pattern, of light and darkness, on an external surface of a subject’s eyeball. The system includes a capturing unit to capture a video of the subject’s eyeball from substantially in front of the subject’s eyeball. The system further includes a processing unit in communication with a memory. The processing unit is configured to identify, using an Artificial Intelligence (AI) model, a first appearance of a breakup pattern in the concentric rings pattern caused by a breakup of a tear film on the external surface of the subject’s eyeball. The processing unit is configured to determine the tear-breakup time based on a timestamp associated with a frame of the video at the first appearance of the breakup pattern and a timestamp associated with a previous blink of a subject’s eyelid, from the captured video.

To further clarify the advantages and features of the present invention, a more particular description of the invention will be rendered by reference to specific embodiments thereof, which are illustrated in the appended drawings. It is appreciated that these drawings depict only typical embodiments of the invention and are therefore not to be considered limiting of its scope. The invention will be described and explained with additional specificity and detail in the accompanying drawings.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 illustrates a system for determining a tear-breakup time to assess a risk for dry eye symptoms, according to an embodiment of the present invention;

FIG. 2 illustrates exemplary stages of processing one or more blink moments for creation of a dataset for training purposes, according to an embodiment of the present invention;

FIG. 3a illustrates an exemplary segment of a binary polar patch with centerlines for computing widths of concentric rings in the binary polar patch at different points, according to an embodiment of the present invention;

FIG. 3b illustrates distances measured from the centerline to upper and lower boundaries of the binary polar patches for assessing the uniformity of the computed ring widths at various points, as referred to FIG. 3a, according to an embodiment of the present invention;

FIG. 3c illustrates a presence of breakup in the polar patches as referred to in FIG. 3b, according to an embodiment of the present invention;

FIG. 3d illustrates a breakup frame identified in a cartesian coordinate plane reconstructed by combining the polar patches, as referred to in FIG. 3c, according to an embodiment of the present invention; and

FIG. 4 illustrates a process flow of a method for determining the tear-breakup time using the system, as referred to in FIG. 1, to assess the risk for dry eye symptoms, according to an embodiment of the present invention.

It should be appreciated by those skilled in the art that any block diagrams herein represent conceptual views of illustrative methods embodying the principles of the present disclosure. Similarly, it will be appreciated that any flow charts, flow diagrams, and the like represent various processes that may be substantially represented in computer-readable medium and so executed by a computer or processor, whether or not such computer or processor is explicitly shown.

### DETAILED DESCRIPTIONOF FIGURES

For the purpose of promoting an understanding of the principles of the present disclosure, reference will now be made to the various embodiments and specific language will be used to describe the same. It will nevertheless be understood that no limitation of the scope of the present disclosure is thereby intended, such alterations and further modifications in the illustrated system, and such further applications of the principles of the present disclosure as illustrated therein being contemplated as would normally occur to one skilled in the art to which the present disclosure relates.

It will be understood by those skilled in the art that the foregoing general description and the following detailed description are explanatory of the present disclosure and are not intended to be restrictive thereof.

Whether or not a certain feature or element was limited to being used only once, it may still be referred to as “one or more features” or “one or more elements” or “at least one feature” or “at least one element.” Furthermore, the use of the terms “one or more” or “at least one” feature or element does not preclude there being none of that feature or element, unless otherwise specified by limiting language including, but not limited to, “there needs to be one or more…” or “one or more elements is required.”

Reference is made herein to some “embodiments.” It should be understood that an embodiment is an example of a possible implementation of any features and/or elements of the present disclosure. Some embodiments have been described for the purpose of explaining one or more of the potential ways in which the specific features and/or elements of the proposed disclosure fulfill the requirements of uniqueness, utility, and non-obviousness.

Use of the phrases and/or terms including, but not limited to, “a first embodiment,” “a further embodiment,” “an alternate embodiment,” “one embodiment,” “an embodiment,” “multiple embodiments,” “some embodiments,” “other embodiments,” “further embodiment”, “furthermore embodiment”, “additional embodiment” or other variants thereof do not necessarily refer to the same embodiments. Unless otherwise specified, one or more particular features and/or elements described in connection with one or more embodiments may be found in one embodiment, or may be found in more than one embodiment, or may be found in all embodiments, or may be found in no embodiments. Although one or more features and/or elements may be described herein in the context of only a single embodiment, or in the context of more than one embodiment, or in the context of all embodiments, the features and/or elements may instead be provided separately or in any appropriate combination or not at all. Conversely, any features and/or elements described in the context of separate embodiments may alternatively be realized as existing together in the context of a single embodiment.

Any particular and all details set forth herein are used in the context of some embodiments and therefore should not necessarily be taken as limiting factors to the proposed disclosure.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises... a” does not, without more constraints, preclude the existence of other devices or other sub-systems or other elements or other structures or other components or additional devices or additional sub-systems or additional elements or additional structures or additional components.

The term “couple” and the derivatives thereof refer to any direct or indirect communication between two or more elements, whether or not those elements are in physical contact with each other. The terms “transmit”, “receive”, and “communicate” as well as the derivatives thereof encompass both direct and indirect communication. The term “or” is an inclusive term meaning “and/or”. The phrase “associated with,” as well as derivatives thereof, refer to include, be included within, interconnect with, contain, be contained within, connect to or with, coupled to or with, be communicable with, cooperate with, interleave, juxtapose, be proximate to, be bound to or with, have, have a property of, have a relationship to or with, or the like. The term “controller” refers to any device, system, or part thereof that controls at least one operation. The functionality associated with any particular controller may be centralized or distributed, whether locally or remotely. The phrase “at least one of,” when used with a list of items, means that different combinations of one or more of the listed items may be used, and only one item in the list may be needed. For example, “at least one of A, B, and C” includes any of the following combinations: A, B, C, A and B, A and C, B and C, and A and B and C, and any variations thereof. As an additional example, the expression “at least one of a, b, or c” may indicate only a, only b, only c, both a and b, both a and c, both b and c, all of a, b, and c, or variations thereof. Similarly, the term “set” means one or more. Accordingly, the set of items may be a single item or a collection of two or more items.

Moreover, multiple functions described below may be implemented or supported by one or more computer programs, each of which is formed from computer-readable program code and embodied in a computer-readable medium. The terms “application” and “program” refer to one or more computer programs, software components, sets of instructions, procedures, functions, objects, classes, instances, related data, or a portion thereof adapted for implementation in a suitable computer-readable program code. The phrase “computer readable program code” includes any type of computer code, including source code, object code, and executable code. The phrase “computer-readable medium” includes any type of medium capable of being accessed by a computer, such as Read Only Memory (ROM), Random Access Memory (RAM), or any other type of memory. A “non-transitory” computer-readable medium excludes wired, wireless, optical, or other communication links that transport transitory electrical or other signals. A non-transitory computer-readable medium includes media where data may be permanently stored and media where data may be stored and later overwritten, such as a rewritable optical disc or an erasable memory device.

Traditional TBUT measurement methods are often subject to human error and artifacts caused by blinking, illumination inconsistencies, or reflections from eyelashes, which can all lead to inaccurate results. By leveraging an Artificial Intelligence (AI)-based non-invasive technique in the present disclosure, such challenges can be minimized or eliminated. An AI model can be trained to recognize the subtle patterns in tear film behavior that indicate true breakup, distinguishing them from optical artifacts or transient disturbances caused by blinks. The AI model can analyze high-definition imaging data, such as videos or images captured by non-invasive devices like infrared cameras or high-resolution video systems, to automatically detect and track the tear film’s stability in real-time.

Therefore, various embodiments of the present disclosure relate to an AI-based non-invasive method and a system for accurately computing tear breakup time based on identifying the tear breakup pattern, ensuring a comfortable experience for the patient. The tear film spreads from a lower eyelid to a corneal surface after a blink forming a continuous surface. The tear film breaks up and the time at which the tear film breaks up indicates the severity of the dry eye condition. Projecting a concentric rings pattern and analyzing the frames from a capturing unit may help in detecting the breakup area and breakup time. By processing large amounts of image data, an AI model can continuously refine the accuracy, adapting to variations in individual tear film characteristics and environmental conditions. Such an approach leads to more reliable and precise Tear Breakup Time (TBUT) measurements, which can be used for diagnosing dry eye disease and monitoring its progression.

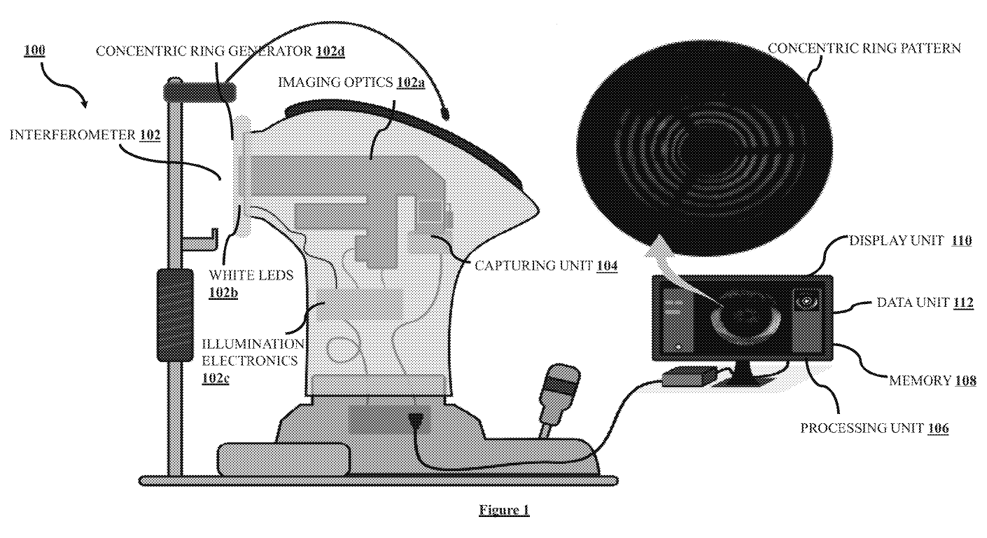

FIG. 1 illustrates a system 100 for determining tear-breakup time (TBUT) to assess a risk for dry eye symptoms, according to an embodiment of the present invention.

Referring to FIG. 1, the system 100 may include an interferometer 102 which may be coupled with an imaging optics 102a, a plurality of light emitting diode (LEDs) 102b, an illumination electronics 102c, a concentric rings generator 102d, and a capturing unit 104. The system 100 may include a processing unit 106, a memory 108, and a display unit 110.

In a non-limiting example, the system 100 may recognize the exact moment when the tear film begins to break up, without the interference of external factors. The system 100 may analyze tear film dynamics, identify breakup patterns, and compute the TBUT with high accuracy.

In an embodiment, the interferometer 102 may measure the thickness and stability of the tear film on the ocular surface by exploiting the principles of light interference. The interferometer 102 may be used for analyzing the tear film’s lipid layer, which plays a crucial role in maintaining the stability of the tear film. The interferometer 102 may operate by emitting light waves onto the tear film surface, and the reflected light from the tear film may create interference patterns based on the thickness of the lipid layer. As the tear film begins to break up, the interference pattern changes, providing real-time data on the stability of the tear film. By capturing subtle changes in the tear film’s behaviour, the interferometer 102 may provide highly accurate and reproducible measurements of the TBUT, essential for diagnosing dry eye conditions. In a non-limiting example, the interferometer 102 may use advanced algorithms to detect the changes in the interference pattern. In an advantageous aspect, the use of the interferometer 102 may ensure reliable, consistent, and objective assessment of tear film dynamics while enhancing patient comfort and reducing clinician variability.

In an embodiment, the imaging optics 102a of the interferometer 102 may be employed to perform precise measurements and analysis of the tear film’s surface and lipid layer by capturing detailed interference patterns. In an implementation, the imaging optics 102a may consist of a combination of high-quality lenses, beam-splitters, and detectors that may work together to direct light using a light source onto the ocular surface and capture the reflected light waves. In a non-limiting example, the light source, such as, but not limited to, a laser, may impart a coherent light. The light source may be directed at the tear film, and the interference patterns may be generated based on the optical path differences between the light reflected from the tear film and surrounding layers. In an implementation, the imaging optics 102a may be designed to focus the light with exceptional clarity and precision to detect subtle variations in the tear film’s structure, ensuring that even minute changes in the lipid layer thickness are accurately captured. In a non-limiting example, the interferometer 102 may employ adaptive imaging optics 102a to correct for aberrations caused by the eye’s curvature or lens imperfections, further improving the accuracy and resolution of the captured interference patterns.

In another embodiment, the plurality of LEDs 102b may be configured in conjunction with the interferometer 102 to illuminate the ocular surface. The plurality of LEDs 102b may be arranged to provide uniform, multi-angle, or wavelength-specific illumination, improving the contrast and resolution of interference patterns observed during the TBUT evaluation. The plurality of LEDs 102b may be selectively activated or modulated to enhance dynamic imaging of tear film disruptions, thereby enabling a more accurate and reproducible assessment of the ocular surface conditions. In an implementation, the plurality of LEDs 102b may emit white light. In an advantageous aspect, the integration of the plurality of LEDs 102b into the interferometer 102 may offer improved flexibility, redundancy, and adaptability for non-invasive clinical diagnostics.

In one embodiment, the illumination electronics 102c may be operatively coupled to the interferometer 102. The illumination electronics 102c may comprise one or more light sources, drivers, control circuitry, and associated optical components configured to emit controlled illumination suitable for interferometric analysis of the tear film. The output of the illumination electronics 102c may be modulated in terms of intensity, wavelength, or temporal characteristics to enhance contrast and resolution in tear film interference patterns. In an advantageous aspect, such features may allow for improved detection sensitivity and repeatability in the TBUT measurements. The configuration of the illumination electronics 102c may be dynamically adjusted based on user input or automated feedback mechanisms to optimize the imaging conditions for various ocular surface characteristics or environmental lighting conditions.

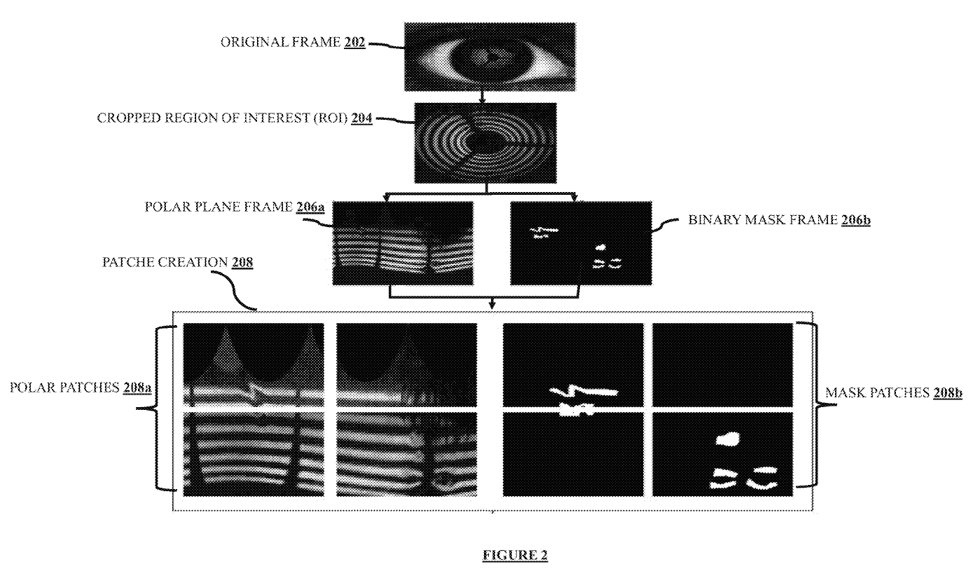

FIG. 2 illustrates different stages of processing the one or more blink moments for the creation of a dataset for training purposes, according to an embodiment of the present invention.

In an embodiment, the interferometer 102 may incorporate the concentric rings generator 102d configured to project structured illumination patterns of light and darkness, such as concentric rings, onto the ocular surface to facilitate the assessment of the TBUT. In an implementation, concentric rings generator 102d of the interferometer 102 may be configured to project a plurality of concentric rings patterns of light and darkness on an external surface of a subject’s eyeball using the concentric rings generator 102d. The concentric rings generator 102d may include one or more optical components, such as diffractive elements, lenses, or spatial light modulators, operatively arranged to produce high-contrast, uniformly spaced ring patterns that enhance the visibility of tear film irregularities. The generated concentric rings may be analyzed via reflected light interference to detect disruptions or breaks in the tear film with high spatial and temporal resolution. The concentric rings generator 102d may function in coordination with the illumination electronics and imaging system to enable real-time or time-lapse visualization of tear film dynamics. In an advantageous aspect, such configuration may allow for more precise and repeatable TBUT measurements, particularly under varying environmental and physiological conditions.

In an embodiment, the capturing unit 104 may be configured to acquire interferometric image data of the ocular surface to evaluate the TBUT. The capturing unit 104 may be configured to capture the video of the subject’s eyeball from substantially in front of the subject’s eyeball. In a non-limiting example, the capturing unit 104 may be configured to capture the video of one or more blink moments of the subject’s eyelid. The video may comprise a plurality of concentric rings pattern frames. The plurality of concentric rings pattern frames captured by the capturing unit 104 may be referred to as original frame 202 as shown in FIG. 2. The capturing unit 104 may comprise one or more imaging sensors, such as but not limited to Charge-Coupled Device (CCD) or Complementary Metal-Oxide-Semiconductor (CMOS) cameras, along with associated optics, filters, and signal processing circuitry. The capturing unit 104 may be positioned in front of the subject’s eyeball to receive reflected light from the tear film surface. In some embodiments, the capturing unit 104 may include onboard or external processing capabilities to support real-time image enhancement, pattern recognition, or automated TBUT calculation.

In an embodiment, the processing unit 106 may be in communication with the memory 108. The processing unit 106 may be configured to initiate or stop one or more routines or a process based on the images or video associated with one or more blink frames received from the capturing device 104. For instance, a region of interest (ROI) 204 may be cropped from the original frame 202 for further processing, as also shown in FIG. 2. The processing unit 106 may be a single processing unit or several units, all of which could include multiple computing units. The processing unit 106 may be implemented as one or more microprocessors, microcomputers, microcontrollers, digital signal processors, central processing units, state machines, logic circuitries, and/or any devices that manipulate signals based on operational instructions. Among other capabilities, the processing unit 106 may be configured to fetch and execute computer-readable instructions and data stored in the memory 108.

In an embodiment, the memory 108 may include any non-transitory computer-readable medium known in the art including, for example, volatile memory, such as static random access memory (SRAM) and dynamic random access memory (DRAM), and/or non-volatile memory, such as read-only memory (ROM), erasable programmable ROM, flash memories, hard disks, optical disks, and magnetic tapes.

In an embodiment, the data unit 112 amongst other things, includes routines, programs, objects, components, data structures, etc., which perform particular tasks or implement data types. The data unit 112 may also be implemented as signal processor(s), state machine(s), logic circuitries, and/or any other device or component that manipulates signals based on operational instructions.

Further, the data unit 112 may be implemented in hardware, instructions executed by a processing unit, or by a combination thereof. The processing unit 106 may comprise a processor, such as the processor, a state machine, a logic array, or any other suitable devices capable of processing instructions. The processing unit 106 may be a general-purpose processor that executes instructions to cause the general-purpose processor to perform the required tasks or, the processing unit 106 can be dedicated to performing the required functions. In another embodiment of the present disclosure, the data unit 112 may be machine-readable instructions (software) that, when executed by the processing unit 106, perform any of the described functionalities.

In an embodiment, the processing unit 106, may be in communication with the memory 108. The processing unit 106 may be configured to identify, using an Artificial Intelligence (AI) model, a first appearance of a breakup pattern in the concentric rings pattern caused by a breakup of a tear film on the external surface of the subject’s eyeballfor assessing the health of a tear film. In an implementation, the processing unit 106 of the system 100 may analyze image data captured during interferometric evaluation of the tear film. In an embodiment, the processing unit 106 may be operatively connected to the capturing unit 104 to receive a sequential frames displaying concentric rings patterns projected onto the ocular surface. The processing unit 106 may be configured to execute one or more algorithms, including the AI model, such as but not limited to a convolutional neural network (CNN), recurrent neural network (RNN), or other machine learning (ML) architecture trained to identify tear film breakup patterns within the concentric rings data. In a non-limiting example, the AI model may detect features such as ring distortion, discontinuity, or irregular reflection indicative of localized or widespread tear film instability. Based on temporal and spatial analysis of the plurality of concentric rings patterns, the processing unit 106 may assess the health of the tear film and determine quantitative or qualitative TBUT metrics. In a non-limiting example, the CNN-based AI model may be preferred for image-based analysis due to CNN’s strong performance in visual pattern recognition tasks. The CNN-based AI model may be trained and developed specifically for the blink frame classification. The CNN-based AI model may distinguish between closed and open eyelids, where frames with fully or partially closed eyelids may be labeled as blink frames, and those with open eyelids may be categorized as non-blink frames. Using such a trained blink detection AI model, blink frames may be accurately identified from video sequences or image streams, enabling automated and reliable blink detection in various applications.

In an implementation, the AI model may be trained based on a dataset of labeled samples obtained from a plurality of training sample subjects for identifying one or more blink frames and one or more non-blink frames within the captured video comprising the multiple concentric rings pattern frames. The blink frames may correspond to the closed eyelids of the subject and the non-blink frames may correspond to the open eyelids of the subject. In another implementation, the AI model may be trained based on a dataset of labeled samples obtained from the plurality of training sample subjects for identifying a first region and a second region in each binary mask frame. The first region may correspond to a breakup region within the tear film and the second region may correspond to a non-breakup region within the tear film. In another implementation, the AI model may be trained based on a dataset of labeled samples obtained from the plurality of training sample subjects for identifying non-uniformity due to the breakup region in each of the created binary mask frame. In another implementation, the AI model may be trained based on a dataset of ground truth label values associated with the breakup pattern.

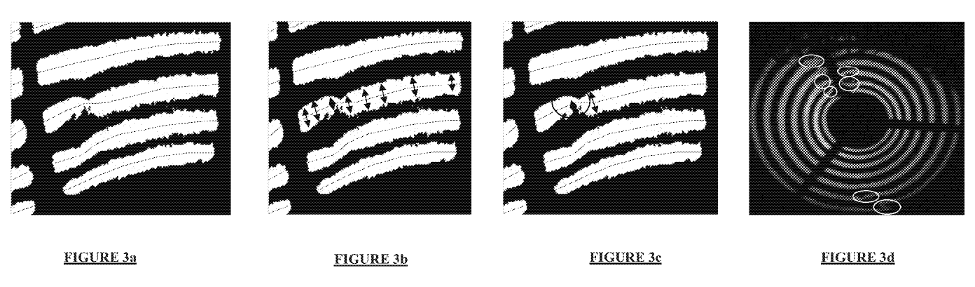

FIG. 3a illustrates an exemplary segment of the binary polar patch with centerlines for computing widths of concentric rings in the binary polar patch at different points, according to an embodiment of the present invention.

FIG. 3b illustrates distances measured from the centerline to upper and lower boundaries of the binary polar patches for assessing the uniformity of the computed ring widths at various points, as referred to in FIG. 3a, according to an embodiment of the present invention.

FIG. 3c illustrates the presence of breakup in the polar patches as referred to in FIG. 3b, according to an embodiment of the present invention.

FIG. 3d illustrates the breakup frame identified in the cartesian coordinate plane reconstructed by combining the polar patches, as referred to in FIG. 3c, according to an embodiment of the present invention.

In an implementation, to identify the breakup pattern based at least on the concentric rings pattern frames, the processing unit 106 may be configured to convert a cartesian plane frame present in ROI 204 associated with each of the concentric rings pattern frames into a corresponding polar plane frame 206a. The processing unit 106 may be configured to further create a binary mask frame 206b of the concentric rings pattern corresponding to each of the polar plane frame 206a. The processing unit 106 may further segment the concentric rings pattern within each of the created polar plane frame 206a and the binary mask frame 206b into a plurality of patches 208. In an implementation, a plurality of polar patches 208a may be obtained corresponding to the polar plane frame 206a and a plurality of mask patches 208b may be obtained corresponding to the binary mask frame 208b. The processing unit 106 may further determine a width of the bright part of the concentric rings pattern with reference to the centerline of each of the bright part of the concentric rings pattern at a plurality of locations within each patch of the binary mask frames, as referred to in FIGS. 3a, 3b, and 3c. In an implementation, to determine the width of the binary polar ring, the system 100 may measure the distance from the centerline to both the upper and lower boundaries of the ring. Such measurement process may be repeated at multiple locations along the entire length of the polar ring segment to ensure comprehensive coverage and accurate characterization of the concentric ring’s geometric profile. Such repeated measurements may enable the assessment of width uniformity and detection of localized variations that may be indicative of tear film irregularities. The processing unit 106 may also determine whether the determined width exceeds a value of a predefined threshold and annotate at least one location associated with a patch with the determined width exceeds the value of the predefined threshold. The annotated location may indicate the presence of the breakup pattern. In a non-limiting example, determining the value of the predefined threshold may be based on an average of the width of the concentric rings pattern. In an implementation, the system 100 may evaluate the uniformity of the computed width of the concentric rings pattern across multiple points along the binary polar ring patch. The non-uniform variations in the width that exceed the value of the predefined threshold may be an indicative of the tear film breakup. In an advantageous aspect, such assessment may enable the detection and classification of breakup patterns by determining whether such variations are present, thereby identifying the presence or absence of tear film instability within the examined ROI.

In an embodiment, the processing unit 106, upon detecting the breakup within the given polar patch frame, may provide the corresponding patch to the AI model for further analysis. The AI model may be configured to localize the precise regions of breakup within the patch and to generate visual annotations highlighting the identified areas. In an implementation, the AI model may produce a segmentation mask in which the breakup regions may be highlighted in white, while non-breakup areas may be rendered in black. In an advantageous aspect, the AI model may enable clear visualization and differentiation of affected regions within the tear film for diagnostic or analytical purposes. After the analysis of all individual polar patches within the single polar frame is completed, the processed patches may be reassembled to reconstruct the full polar frame. In an advantageous aspect, the reconstituted frame may retain the spatial relationships of the original patches, allowing for accurate localization of identified breakup regions. A boundary extraction technique may be applied to the full polar frame for determining one or more coordinates of the breakup locations, enabling precise mapping of tear film discontinuities across the ocular surface.

In an embodiment, the processing unit 106, upon identifying the breakup locations, may send a signal to the display unit 110 to highlight the breakup on the reconstructed polar plane frame to facilitate localization of tear film instability. The annotated polar plane frame may be further converted back into the cartesian coordinate form to correspond with the original spatial orientation of the ocular surface. Based on such transformation, the breakup frame may be confirmed, as shown in FIG. 3d. The system 100 may utilize the timestamp associated with the most recent blink frame, in conjunction with the timestamp of the identified breakup frame, to compute the TBUT in seconds. In an advantageous aspect, the timestamp value may serve as a quantitative indicator of tear film health and stability.

In an embodiment, the processing unit 106 may correlate a timestamp associated with the breakup pattern and a timestamp associated with a previous blink moment of the subject’s eyelid from the captured video. In an implementation, the correlation for determining the ocular tear-breakup time may be based on a difference between the timestamp associated with the frame of the video at the first appearance of the breakup pattern and the timestamp associated with the previous blink moment of the subject’s eyelid from the captured video.

In an embodiment, the processing unit 106, may further determine the ocular tear-breakup time based on the correlation to assess the risk for dry eye symptoms, wherein the ocular tear-breakup time less than a value of a predefined criteria indicates the risk for dry eye symptoms. In a non-limiting example, if the determined TBUT is less than equals to 5, the risk may be considered as severe, whereas, if the determined TBUT is between 5 and 10, the risk may be considered as moderate, whereas, if the determined TBUT is greater than equals to 10, the risk may be considered as normal. The results may then be stored in the memory 108, displayed on the display unit 110, or transmitted for diagnostic interpretation or integration into broader ocular health assessment workflows. Thus, the determined TBUT may provide critical information regarding the stability of the tear film by recording the time interval between the complete blink and the first appearance of tear film disruption. A shorter TBUT may indicate an unstable tear film, which may be a key marker of the dry eye conditions. The TBUT measurement may help in identifying the presence and severity of the dry eye, distinguishing between different subtypes such as evaporative and aqueous-deficient dry eye, and monitoring the effectiveness of treatment over time.

In a non-limiting example, the AI model may be trained to determine the predetermined criteria based on a dataset of ground truth label values associated with the dry eye conditions.

In an embodiment, the various units/components such as the memory 108 and the data unit 112 may be a part of the processing unit 106. In another embodiment, the processing unit 106 may be configured to identify the breakup pattern, correlate the timestamps associated with the breakup pattern and the previous blink moment from the captured video, and determine the ocular tear-breakup time based on the correlation to assess the risk for dry eye symptoms. The data unit 136 may serve, amongst other things, as a repository for storing data processed, received, and generated signals from modules 102-104, the processing unit 106, and the memory 108.

In one embodiment, the display unit 110, operatively coupled with the interferometer 102, may be configured to present visual output related to the TBUT measurements. The display unit 110 may be, but is not limited to, a monitor, touchscreen, a head-mounted display, or an integrated graphical interface, and may be designed to render real-time or recorded interferometric images, processed data, and diagnostic overlays. The display unit 110 may provide a visual representation of interference patterns, TBUT intervals, tear film dynamics, and other relevant metrics, enabling clinicians or users to interpret the ocular surface condition efficiently. In some configurations, the display unit 110 may also support user interaction, allowing manual adjustment of system parameters, review of time-lapse sequences, or integration with patient records. The coupling between the display unit 110 and the interferometer 102 may be wired or wireless, depending on the application, and may include data from the capturing unit 104, image processing module, and analysis algorithms for enhanced visualization and usability in clinical or point-of-care environments.

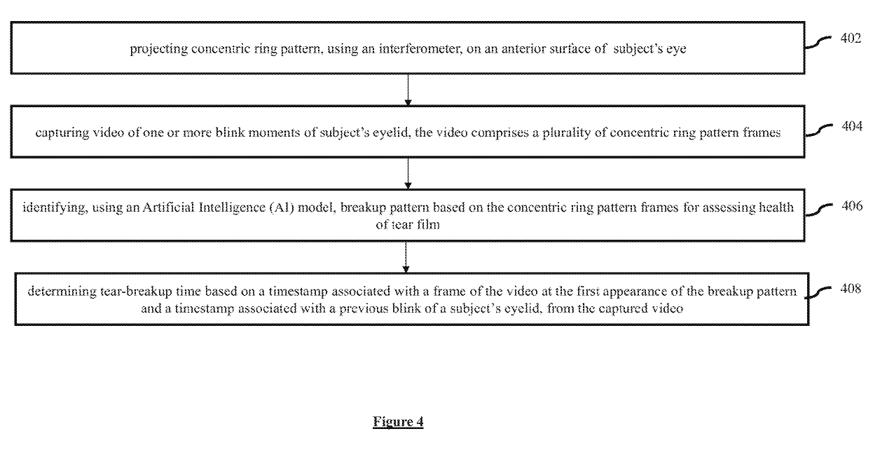

FIG. 4 illustrates a process flow of a method 400 for determining the tear-breakup time using the system 100, as referred to in FIG. 1, to assess the risk for dry eye symptoms, according to an embodiment of the present invention.

In an embodiment, at step 402, the interferometer 202 may project the concentric rings pattern of light and darkness on the external surface of the subject’s eyeball by using the concentric rings generator 102d. In a non-limiting example, the concentric rings generator 102d may generate two types of concentric rings patterns, that is a Keratograph-like pattern and a quadrant-based circular pattern. The Keratograph-like pattern may be characterized by uniformly spaced circular rings that provide a detailed map of the corneal curvature whereas in the quadrant-based circular pattern, the rings may be segmented into quadrants to allow for localized analysis of the corneal surface. Both the patterns may enable precise corneal topography assessment in various ophthalmic and optometric applications.

In an embodiment, at step 404, the capturing unit 104 may capture the video of the subject’s eyeball from substantially in front of the subject’s eyeball. In an implementation, the video may comprise one or more blink moments that may be captured for at least 10 seconds or more. The minimum time frame of 10 seconds may be essential for conducting a reliable risk assessment. The capturing unit 104 may record high-resolution images or video sequences of interference patterns generated by the tear film. These interference patterns may provide critical visual cues for detecting tear film instability, such as localized thinning or complete break-up. The capturing unit 104 may operate in synchronization with the illumination electronics 102c and the concentric rings generator 102d to ensure temporal alignment and enhance the accuracy of TBUT analysis.

In an embodiment, at step 406, the processing unit 106 may identify the first appearance of the breakup pattern in the concentric rings pattern caused by the breakup of the tear film on the external surface of the subject’s eyeball using the AI model. In an implementation, multiple video samples may be collected for preparing a comprehensive dataset. From each video, individual frames may be extracted, and the ROI may be isolated using the AI-based segmentation model designed for extracting the ROI.

In an embodiment, at step 408, the processing unit 106 may determine the tear-breakup time based on the timestamp associated with the frame of the video at the first appearance of the breakup pattern and the timestamp associated with the previous blink of the subject’s eyelid, from the captured video. In an implementation, the determining of the tear breakup may be based on the correlation which may include finding the difference between the timestamp associated with the frame of the video at the first appearance of the breakup pattern and the timestamp associated with the previous blink moment of the subject’s eyelid from the captured video. The segmentation model may produce binary mask frames 206b corresponding to the polar plane frames 206a. In each of the binary mask frames 206b, the breakup area may be highlighted in white, while the rest of the frame remains black. Both the polar plane frames 206a and their corresponding binary mask frames 206b may be further divided into smaller patches 208a and 208b to enhance learning efficiency during the AI model training. To avoid overfitting caused may be due to an excessive number of patches, the AI model may be trained to focus only on the white region in each mask patch 208b. To precisely locate tear film breakup, the coordinates of the breakup regions may be identified using the boundary extraction technique applied to the full polar binary frame. These coordinates may be mapped back to the corresponding locations on the polar plane frame 206a.

In an embodiment, the tear-breakup time less than the value of the predefined criteria may indicate the risk for dry eye symptoms. In an implementation, the timestamps of the blink frame and the breakup frame may be used to calculate the breakup time in seconds by subtracting the blink frame timestamp from that of the breakup frame.

The present disclosure offers several advantages, especially in terms of usability, simplicity, and efficiency. Here are some of the key benefits:
Improved Accuracy and Consistency: The disclosed method 400 and the system 100 can consistently identify tear film break-up patterns with high accuracy by using AI model. Unlike manual assessments, which are subject to human error and variability between clinicians, the use of the AI model helps in providing objective and repeatable measurements.
Faster Analysis: Through the disclosed method 400 and the system 100, the time needed to analyze the video sequences can be significantly reduced for automated TBUT identification. By using the AI model in the present disclosure, the assessment may be accomplished in a few seconds, allowing for faster diagnosis and streamlined workflows.
Scalability: Through the disclosed method 400 and the system 100, a large volume of patient data may be assessed quickly through the use of the AI model, making the present method 400 and the system 100 ideal for high-throughput environments such as research studies, clinical trials, or telemedicine platforms. Such scalability can also support broader screening and monitoring efforts.
Early Detection of Dry Eye Disease (DED): By precisely identifying the moment of tear film break-up, the disclosed method 400 and the system 100 can detect subtle signs of dry eye disease earlier than traditional methods. This allows for earlier intervention and better patient outcomes.
Reduced Subjectivity: Traditional TBUT assessments can vary depending on the clinician’s experience and interpretation. However, the present disclosure can eliminate such subjectivity, providing standardized results across different users and clinical settings.
Support for Remote and Automated Screening: The disclosed method 400 and the system 100 can be integrated into remote diagnostic platforms, enabling dry eye screening in telehealth settings or underserved regions without the constant need for a specialist.
Enhanced Training and Decision Support: Clinicians can use the disclosed method 400 and the system 100 for AI-generated TBUT analysis as a reference or second opinion, supporting better decision-making and serving as an educational tool for training new practitioners.

While specific language has been used to describe the disclosure, any limitations arising on account of the same are not intended. As would be apparent to a person in the art, various working modifications may be made to the method in order to implement the inventive concept as taught herein.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein.

What is claimed is:

1. A method for determining tear-breakup time, the method comprising:
projecting a concentric rings pattern, of light and darkness, on an external surface of a subject’s eyeball, using a concentric rings generator of light and darkness;
capturing a video of the subject’s eyeball from substantially in front of the subject’s eyeball;
identifying , using an Artificial Intelligence model, a first appearance of a breakup pattern in the concentric rings patterns, caused by a breakup of a tear film on the external surface of the subject’s eyeball; and
determining the tear-breakup time based on a timestamp associated with a frame of the video at the first appearance of the breakup pattern and a timestamp associated with a previous blink of a subject’s eyelid, from the captured video.

2. The method as claimed in claim 1, wherein identifying the first appearance of the breakup pattern in the concentric rings pattern comprises:
converting a cartesian plane frame associated with each of the concentric rings pattern into a corresponding polar plane frame;
creating a binary mask frame of the concentric rings pattern corresponding to each polar plane frame;
segmenting the concentric rings pattern within each binary mask frame created into a plurality of patches;
determining a width of the bright part of the concentric rings pattern with reference to a centerline of each bright part of the concentric rings pattern at a plurality of locations within each patch of the binary mask frame;
determining whether the determined width exceeds a value of a predefined threshold; and
annotating at least one location associated with a patch with the determined width exceeding the value of the predefined threshold, wherein the location indicates a presence of the breakup pattern.

3. The method as claimed in claim 2, wherein determining the value of the predefined threshold is at least based on an average of the width of the concentric rings pattern.

4. The method as claimed in claim 1, wherein the Artificial Intelligence model is trained at least based on:
a dataset of labeled samples obtained from a plurality of training sample subjects for identifying one or more blink frames and one or more non-blink frames within the captured video, wherein the blink frames correspond to closed eyelids of the subject and the non-blink frames correspond to open eyelids of the subject;
a dataset of labeled samples obtained from the plurality of training sample subjects for identifying a first region and a second region in each binary mask frame, wherein the first region corresponds to a breakup region within the tear film and the second region corresponds to a non-breakup region within the tear film;
a dataset of labeled samples obtained from the plurality of training sample subjects for identifying non-uniformity due to the breakup region in each of the binary mask frame, and
a dataset of ground truth label values associated with the breakup pattern.

5. The method as claimed in claim 1, wherein determining the tear-breakup time is based on a difference between the timestamp associated with the frame of the video at the first appearance of the breakup pattern and the timestamp associated with the previous blink of the subject’s eyelid, from the captured video.

6. A system for determining tear-breakup time, the system comprising:
a concentric rings generator of light and darkness configured to project a concentric rings pattern, of light and darkness, on an external surface of a subject’s eyeball;
a capturing unit to capture a video of the subject’s eyeball from substantially in front of the subject’s eyeball;
a processing unit in communication with a memory, the processing unit configured to:
identify, using an Artificial Intelligence model, a first appearance of a breakup pattern in the concentric rings pattern caused by a breakup of a tear film on the external surface of the subject’s eyeball; and
determine the tear-breakup time based on a timestamp associated with a frame of the video at the first appearance of the breakup pattern and a timestamp associated with a previous blink of a subject’s eyelid, from the captured video.

7. The system as claimed in claim 6, wherein to identify the first appearance of the breakup pattern in the concentric rings pattern, the processing unit is configured to:
convert a cartesian plane frame associated with each of the concentric rings pattern into a corresponding polar plane frame;
create a binary mask frame of the concentric rings pattern corresponding to each polar plane frame;
segment the concentric rings pattern within each binary mask frame created into a plurality of patches;
determine a width of the bright part of the concentric rings pattern with reference to a centerline of each bright part of the concentric rings pattern at a plurality of locations within each patch of the binary mask frame;
determine whether the determined width exceeds a value of a predefined threshold; and
annotate at least one location associated with a patch with the determined width exceeds the value of the predefined threshold, wherein the location indicates a presence of the breakup pattern.

8. The system as claimed in claim 7, wherein the processing unit is configured to determine the value of the predefined threshold is at least based on an average width of the bright part of each concentric ring.

9. The system as claimed in claim 6, wherein the Artificial Intelligence model is trained at least based on:
a dataset of labeled samples obtained from a plurality of training sample subjects to identify one or more blink frames and one or more non-blink frames within the captured video, wherein the blink frames correspond to closed eyelids of the subject and the non-blink frames correspond to open eyelids of the subject;
a dataset of labeled samples obtained from the plurality of training sample subjects to identify a first region and a second region in each binary mask frame, wherein the first region corresponds to a breakup region within the tear film and the second region corresponds to a non-breakup region within the tear film;
a dataset of labeled samples obtained from the plurality of training sample subjects to identify non-uniformity due to the breakup region in each of the binary mask frame, and
a dataset of ground truth label values associated with the breakup pattern.

10. The system as claimed in claim 6, wherein the tear-breakup time is determined based on a difference between the timestamp associated with the frame of the video at the first appearance of the breakup pattern and the timestamp associated with the previous blink of the subject’s eyelid, from the captured video.
